(19) United States
(12) Patent Application Publication
(10) Pub. No.: US 2010/0310650 A1
(43) Pub. Date: Dec. 9, 2010

(54) Pharmaceutical composition for the oral administration of omega polyenoic fatty acids and one or more active principles incompatible therewith, and a process for its preparation

(76) Inventor: Roberto Valducci, Savignano Sul Rubicone (IT)

Correspondence Address: STETINA BRUNDA GARRED & BRUCKER, 75 ENTERPRISE, SUITE 250, ALISO VIEJO, CA, 92656, US

(21) Appl. No.: 12/308,414

(22) PCT Filed: Jun. 26, 2007
(86) PCT No.: PCT/EP2007/056344

§ 371 Date: Dec. 11, 2008

(51) Int. Cl. A61K 31/20; A61K 9/48; A61K 31/202; A61K 31/366; A61K 31/616; A61K 31/355; A61K 9/52; A61P 7/02; A61P 7/00; B05D 1/02

(52) U.S. Cl. 424/452; 424/463; 514/560; 424/456; 514/460; 514/163; 514/458; 424/457; 427/2.14

(57) ABSTRACT

A pharmaceutical composition for the oral administration of omega polyenoic fatty acids combined with one or more active principles incompatible therewith, is described; also described is a process for preparing said pharmaceutical composition.

### FIELD OF THE INVENTION

The present invention relates to the field of pharmaceutical compositions, and in particular to a new pharmaceutical composition for the oral administration of omega polyenoic fatty acids and one or more active principles incompatible therewith.

### STATE OF THE ART

Omega polyenoic fatty acids are long chain polyunsaturated fatty acids comprising between 18 and 22 carbon atoms. Of these, omega-3 polyenoic acids, in which the first unsaturated bond is between the third and fourth carbon atoms counting from the terminal methyl group, and omega-6 polyenoic acids, in which the first unsaturated bond is between the sixth and seventh carbon atoms, are essential fatty acids.

The most widespread of the omega-3 polyenoic fatty acids, of which fish oil is a rich source, are eicosapentanoic acid, generally known by the acronym EPA, and docosahexanoic acid, known by the acronym DHA. Another important omega-3 polyenoic acid, not of marine but of plant origin, is alpha linolenic acid, known by the acronym ALA.

It is now widely established that omega polyenoic fatty acids compete with arachidonic acid for binding to the enzymes cycloxygenase and lipoxygenase, causing a reduction in blood triglyceride levels. They possess an anti-aggregation and antithrombotic action due to their effect on reducing thromboxane A2 synthesis, and also promote vasodilation and increase bleeding time.

By virtue of their valuable biological effects, omega polyenoic fatty acids are indicated for treating relapses after angioplasty and for reducing angina attacks, as well as for treating hypertriglyceremia when combined with modified dietary regimens or when the response to diet and other non-pharmacological measures alone has proved inadequate.

In the vast majority of cases food sources of omega polyenoic fatty acids are not in fact sufficient to achieve an efficient therapeutic effect, and these acids have to be consumed in the form of pharmaceutical compositions.

The consumption of omega polyenoic fatty acids can enable cholesterol lowering drugs such as statins e.g. simvastatin to function more effectively; said fatty acids can also enhance the effects of blood-thinning drugs such as platelet anti-aggregants e.g. acetylsalicylic acid.

The possibility of physically combining, in the same formulation, omega polyenoic fatty acids with one or more active principles incompatible therewith, such as simvastatin or acetylsalicylic acid, has so far been thought unachievable due to the incompatibility of the components which would give rise to the formation of degradation substances.

As far as the Applicant's knowledge extends, products that comprise these active principles in a single composition have not yet been produced, but instead, in order to gain the benefits of their combination, recourse has had to be made to the administration of several formulations, each containing only one active principle. Instead, the advantages of being able to provide a single composition for the simultaneous oral administration of omega polyenoic fatty acids with active principles incompatible therewith, especially in relation to patient compliance, are evident. The need was therefore felt to provide a composition of this type.

### SUMMARY OF THE INVENTION

The Applicant has now found that a new pharmaceutical composition can be achieved which contains omega polyenoic fatty acids and one or more active principles incompatible therewith, such as statins or platelet anti-aggregants in the same dosage unit, thus allowing their simultaneous administration. The new pharmaceutical composition thus formulated is highly stable, and allows the active principles at various dosages to be orally administered in a single capsule.

The present invention therefore provides a pharmaceutical composition for the oral administration of omega polyenoic fatty acids and one or more active principles incompatible therewith, comprising a capsule containing one or more blended omega polyenoic fatty acids, and a film coating comprising one or more of said active principles incompatible therewith and one or more suitable film-forming agents, possibly mixed with at least one inert substance.

A further aspect of the invention are two processes for preparing the aforesaid pharmaceutical composition.

Characteristics and advantages of the invention will be illustrated in detail in the following description.

### DETAILED DESCRIPTION OF THE INVENTION

The two active principles present in the pharmaceutical composition of the invention—fatty acids on the one hand and one or more active principles incompatible therewith on the other—are maintained separate from one another to within the same dosage unit. In particular, the active principle present in the higher dosage i.e. the fatty acid, is encapsulated, for example in a soft or hard gelatin capsule, while the second active principle, possibly mixed with one or more other active principles, is uniformly distributed around the capsule by means of one of the two preparation processes described hereinafter.

In accordance with the invention, the present compositions typically contain from 100 to 1500 mg of fatty acids, preferably around 1000 mg, within the capsule. Said fatty acids are preferably chosen from the group consisting of omega-3 polyenoic fatty acids, omega-6 polyenoic fatty acids and blends thereof, more preferably being omega-3 polyenoic fatty acid blends.

Particularly preferred in accordance with the invention are omega-3 polyenoic fatty acid blends containing EPA and DHA in a quantity between 20 and 98% by weight on the total weight of the blend, and preferably in quantities equal to at least 60% by weight. In said blends the weight ratio of EPA to DHA is for example between 0.05 and 2.5, preferably between 0.9 and 1.5.

The second active principle—or active principles if more than one—is present in the composition of the invention in variable quantities depending on the active principle, for example in a quantity between 10 and 160 mg for simvastatin and in a quantity between 10 and 300 mg for acetylsalicylic acid, although different dosages can be established in accordance with the invention to hence obtain excellent results in terms of composition stability.

In accordance with the invention, “active principles incompatible with omega polyenoic fatty acids” are specifically platelet anti-aggregants, preferably acetylsalicylic acid, and statins, preferably simvastatin. Particularly preferred are the compositions of the invention which comprise omega polyenoic fatty acids and acetylsalicylic acid or omega polyenoic fatty acids and simvastatin, alone or mixed with a further active principle incompatible with said fatty acids, chosen for example from the group consisting of butyl hydroxyanisole, citric acid, vitamin E, ascorbic acid and mixtures thereof. In accordance with the invention, simvastatin can be used in the present composition in the form of a pure raw material or in microencapsulated form comprising from 10 to 90% of simvastatin.

The second active principle—or active principles if more than one—is applied onto the capsule with one or more film-forming agents and possibly one or more inert substances, making use of suitable solvents. One or more further film coatings free of said active principles and comprising one or more suitable film-forming agents, possibly mixed with at least one inert substance, can be applied onto the film coating containing the second active principle.

Of the film-forming agents of possible use in accordance with the invention, film-forming agents chosen from hydroxypropyl methyl cellulose, polyvinylpyrrolidone, polyvinyl alcohol-polyethylene glycol copolymers, basic polymethacrylate such as the product known by the commercial name Eudragit® E, and mixtures thereof are preferred, while the possible inert substance is chosen for example from talc and lactose monohydrate.

Regarding the quantity of film-forming agent in the present compositions, in the case of statins, the weight ratio of film-forming agent to statin is preferably 0.5:5, whereas in the case of acetylsalicylic acid the weight ratio of film-forming agent to acid is preferably 1:0.5.

Regarding the inert substance, if present, the quantities used in accordance with the invention are as aforestated for the film-forming agent.

The present pharmaceutical compositions can be prepared by a preparation process which is also an aspect of the invention and comprises the following steps:

i) preparing the capsule containing one or more blended omega polyenoic fatty acids;
ii) preparing a solution or suspension comprising at least one film-forming agent and a suitable solvent, the second active principle or a mixture of active principles incompatible with said fatty acids, and possibly one or more inert substance;
iii) applying the film coating onto the capsule derived from step i), by nebulizing the solution or suspension prepared in step ii) with techniques and equipment commonly used in the field of pharmaceutical formulations.

Alternatively, the present pharmaceutical compositions can be prepared by a second preparation process, which is also an aspect of the invention and comprises the following steps:

i′) preparing the capsule containing one or more blended omega polyenoic fatty acids;
ii′) preparing a solution or suspension comprising at least one film-forming agent and a suitable solvent, the second active principle or a mixture of active principles incompatible with said fatty acids, and possibly one or more inert substance;
iii′) possibly mixing said second active principle or a mixture of active principles in is powder form with the inert substance possibly present;
iv′) applying the film coating onto the capsule derived from step i′) by means of several alternate phases of nebulizing the solution or suspension prepared in step ii′) and distributing the second active principle or active principle mixture in powder form, possibly mixed with the inert substance as prepared in step iii′).

In both the preparation processes of the invention, “suitable solvents” means preferably a solvent chosen from acetone, isopropyl alcohol and mixtures thereof if the second active principle is simvastatin, whereas if the second active principle is acetylsalicylic acid, “suitable solvents” means preferably a solvent chosen from water, ethanol and mixtures thereof.

In accordance with the invention, said solvents can possibly be mixed with a buffer at pH 4-8, chosen for example from an acetone buffer or a phosphate buffer.

In a preferred embodiment of the invention, in steps ii) and ii') of the present processes the film-forming agent is firstly dissolved in the pre-selected solvent, then mixed with the other components in solution or suspension. The quantity of film-forming agent used is for example between 1 and 20% by weight on the total weight of the capsule, preferably in a quantity of 2%, while the inert substance, if present, can be added for example in a quantity between 2 and 30% by weight on the total weight of the capsule, preferably in a quantity of 5%.

In addition to the various active principles, coating components and aforementioned solvents, the pharmaceutical compositions in accordance with the invention can also comprise excipients, and/or pharmaceutically acceptable diluents chosen from those conventionally used in pharmaceutical compositions in order to produce a composition suitable for oral administration.

The pharmaceutical compositions prepared as aforedescribed are compositions that immediately release the active principle, but they can also be subjected to further treatments to obtain gastroresistant compositions or modified-release compositions.

To produce gastroresistant compositions, for example, the composition prepared as aforedescribed is subjected to the application, by known techniques, of a further coating with agents sensitive to pH variations, such as methacrylic acid derivatives known by the commercial names Eudragit® L, S and FS, hydroxypropyl methyl cellulose succinate, hydroxymethyl cellulose phthalate, cellulose acetate phthalate, and the like, or mixtures thereof. The aforementioned substances are plasticized with triethyl citrate or the like, in the quantities suggested by the manufacturers of film-forming agents.

To produce modified-release compositions, on the other hand, the composition prepared as aforedescribed is subjected to the application, by known techniques, of a further coating with agents not sensitive to pH variations, such as ethyl cellulose, cellulose acetate butyrate, methacrylic acid derivatives known by the commercial name Eudragit® RS and RL, Shellac and the like.

The following non-limiting examples are given by way of illustration of the present invention.

### Example 1

1 Kg of soft gelatin capsules were prepared, corresponding to about 1,400 capsules, containing 500 mg of omega-3 polyenoic fatty acids per capsule with a minimum EPA-DHA content of 30%.

Said capsules are placed in a bowl equipped with an automated spray, a product and air inlet temperature control system, and nebulized with a solution having the following composition:

| | |
|---|---|
| 10% Eudragit E in Acetone | 250.0 g |
| Acetone | 750.0 g |
| Simvastatin | 16.7 g |
| Talc | 20.0 g |

Production was carried out setting the equipment with the following operative parameters:

| | | |
|---|---|---|
| Basin speed: | 8 | rpm |
| Air inlet temperature: | 50° | C. |
| Product temperature: | 25-30° | C. |
| Nebulizer pressure: | 1 | bar |

The capsules obtained were analysed using the USP apparatus 2 (Paddle Apparatus), obtaining the following results:

| | |
|---|---|
| Degradation products: | <1% |
| Simvastatin content per capsule: | 10 mg |
| Release of simvastatin: | within 30 minutes |

### Example 2

Under the same operative conditions described in example 1, a solution with the following composition:

| | | |
|---|---|---|
| 10% polyvinylalcohol (PVA)-polyethyleneglycol (PEG) | 1,000.0 | g |
| copolymer in 50:50 acetate/isopropyl alcohol buffer | | |
| Simvastatin | 16.7 | g |
| Isopropyl alcohol | 300.0 | g |

is was nebulized onto 1 Kg of hard gelatin capsules containing about 500 mg of the fatty acid mixture of example 1.

The capsules obtained were analysed using the method aforegiven in example 1 to determine the quantity of degradation products and simvastatin content, obtaining results similar to those of example 1.

### Example 3

Under the same operative conditions described in example 1, a solution with the following composition:

| | |
|---|---|
| 10% Eudragit E in Acetone | 500.0 g |
| Acetone | 750.0 g |
| Simvastatin | 16.7 g |
| Talc | 40.0 g |

was nebulized onto 1 Kg of soft gelatin capsules containing about 500 mg of the fatty acid mixture of example 1.

The capsules obtained were analysed using the method aforegiven in example 1 to determine the quantity of degradation products and simvastatin content, obtaining results similar to those of example 1.

### Example 4

Under the same operative conditions aforedescribed in example 1, a solution with the following composition:

| | | |
|---|---|---|
| 10% PVA-PEG copolymer in 50:50 | 1,000.0 | g |
| acetate/isopropyl alcohol buffer | | |
| Simvastatin | 16.7 | g |
| Isopropyl alcohol | 300.0 | g |
| Acetate buffer pH 4.0 | 300.0 | g |

was nebulized onto 1 Kg of soft gelatin capsules containing about 500 mg of the fatty acid mixture of example 1.

The capsules obtained were analysed using the method aforegiven in example 1 to determine the quantity of degradation products and simvastatin content, obtaining results similar to those of example 1.

### Example 5

Under the same operative conditions aforedescribed in example 1, a solution with the following composition:

| | |
|---|---|
| 10% Eudragit E in Acetone | 500.0 g |
| Acetone | 750.0 g |
| Simvastatin | 33.4 g |
| Talc | 40.0 g |

was nebulized onto 1 Kg of soft gelatin capsules containing about 500 mg of the fatty acid mixture of example 1.

The capsules obtained were analysed using the method aforegiven in example 1, obtaining the following results:

| | |
|---|---|
| Degradation products: | <1% |
| Simvastatin content per capsule: | 20 mg |
| Release of simvastatin: | within 30 minutes |

### Example 6

Under the same operative conditions aforedescribed in example 1, a solution with the following composition:

| | |
|---|---|
| 10% Eudragit E in Acetone | 500.0 g |
| Acetone | 750.0 g |
| Simvastatin | 66.8 g |
| Talc | 40.0 g |

was nebulized onto 1 Kg of soft gelatin capsules containing about 500 mg of the fatty acid mixture of example 1.

The capsules obtained were analysed using the method aforegiven in example 1, obtaining the following results:

| | |
|---|---|
| Degradation products: | <1% |
| Simvastatin content per capsule: | 40 mg |
| Release of simvastatin: | within 30 minutes |

### Example 7

Under the same operative conditions aforedescribed in example 1, 27.4 mg of a is solution of 10% PVP in isopropyl alcohol was nebulized onto 1 Kg of soft gelatin capsules containing about 500 mg per capsule of the fatty acid mixture of example 1 followed by application of 17 g of simvastatin, having previously been mixed with 100 g of lactose monohydrate in a suitable high velocity mixer for about 1 minute. The wetting/drying operation was repeated until the powder mix was used up. The capsules obtained were analysed using the method aforegiven in example 1, and the quantity of degradation products was found to be less than 1%.

### Example 8

Under the same operative conditions described in example 1, a solution with the following composition:

| | |
|---|---|
| 20% PVP in isopropyl alcohol | 100.0 g |
| Isopropyl alcohol | 100.0 g |
| 10% Eudragit E in isopropyl alcohol | 200.0 g |

was nebulized onto 1 Kg of soft gelatin capsules containing about 500 mg per capsule of the fatty acid mixture of example 1.

17 g of simvastatin, having been previously mixed with 150 g of lactose monohydrate in a suitable high velocity mixer for 1 minute, were then applied. The wetting/drying operation was repeated until the powder mix was used up. The capsules obtained were analysed using the method aforegiven in example 1, and the quantity of degradation products was found to be less than 1%.

### Example 9

Under the same operative conditions described in example 1, a solution with the following composition:

| | |
|---|---|
| 20% PVP in isopropyl alcohol | 50.0 g |
| Isopropyl alcohol | 50.0 g |
| 10% Eudragit E in isopropyl alcohol | 100.0 g |

was nebulized onto 1 Kg of soft gelatin capsules containing about 500 mg per capsule of the fatty acid mixture of example 1.

18 g of simvastatin, having been previously mixed with 20 g of lactose monohydrate in a suitable high velocity mixer for 1 minute, were then applied. The wetting/drying operation was repeated until the powder mix was used up. The capsules obtained were analysed using the method aforegiven in example 1, and the quantity of degradation products was found to be less than 1%.

### Example 10

About 600 g of a 10% PVA-PEG copolymer suspension in a 50:50 ethanol:water mixture were nebulized onto 1 Kg of soft gelatin capsules, of average weight about 700 mg and containing about 500 mg of the fatty acid mixture of example 1 per capsule, placed in a basin equipped with systems for automated spraying and product temperature and inlet temperature controls, with the purpose of isolating the initial core before applying acetylsalicylic acid under the following operative conditions:

| | | |
|---|---|---|
| Air inlet temperature: | 50-55° | C. |
| Product temperature: | 22-30° | C. |
| Pressure of nebulizer: | 1 | bar |
| Pump speed | 10 | rpm |
| Nozzle diameter | 1 | mm |

Under the same operative conditions, a suspension containing acetylsalicylic acid having the following composition:

| | |
|---|---|
| 10% PVA-PEG copolymer in a 50:50 | 714.0 g |
| ethanol:water mixture | |
| Acetylsalicylic acid | 157.0 g |
| Ethanol | 779.0 g |

was nebulized onto these capsules. White, homogeneous, coated capsules of perfect appearance were thus obtained, containing 100 mg of acetylsalicylic acid per dose in the surface layer and 500 mg of the aforesaid fatty acid mixture inside the capsule.

Said capsules were again placed in a basin in order to render them gastroresistant, using 800 g of an aqueous solution of 15% Eudragit L100-55. The capsules thus obtained were analysed and found to be gastroresistant, to demonstrating the following release profile:

| | |
|---|---|
| 2 hours in 0.1 N HCl: | 0% |
| 1 hour at pH 6.8: | 92% |

### Example 11

Under the same operative conditions described in example 7, a suspension containing acetylsalicylic acid with the following composition:

| | |
|---|---|
| 10% PVA-PEG copolymer in a 50:50 | 714.0 g |
| ethanol:water mixture | |
| acetylsalicylic acid | 157.0 g |
| ethanol | 779.0 g |

was nebulized onto 1 Kg of soft gelatin capsules with an average weight of about 700 mg containing about 500 mg per capsule of the fatty acid mixture of example 1

White, homogeneous, immediate-release capsules with perfect appearance were thus obtained, containing 100 mg of acetylsalicylic acid per dose in the surface layer and 500 mg of the aforesaid fatty acid mixture inside the capsule.

### Example 12

Under the same operative conditions aforedescribed in example 1, 55 mg of a solution of 10% PVP in isopropyl alcohol were nebulized onto 1 Kg of soft gelatin capsules containing about 500 mg per capsule of the fatty acid mixture of example 1 followed by application of 170 g microencapsulated 10% simvastatin having previously been mixed with 20 g of lactose monohydrate in a suitable high velocity mixer for about 1 minute. The wetting/drying operation was repeated until the powder mix was used up.

The capsules obtained were analysed using the method aforegiven in example 1: the quantity of degradation products was found to be less than 1% and the content of simvastatin was 10 mg per capsule.

### Example 13

Under the same operative conditions as example 1, 1.100 kg of a solution of 5% HPMC P-50 in 70:30 acetone:ethanol were nebulized onto 2 Kg of soft gelatin capsules containing about 500 mg per capsule of the fatty acid mixture of example 1 with the purpose of rendering the capsules gastroresistant.

### Example 14

Under the same operative conditions aforedescribed in example 1, 55 g of a 10% PVP solution in isopropyl alcohol were nebulized onto 1 Kg of gastroresistant capsules prepared as aforedescribed in example 13, followed by application of 170 g of 10% microencapsulated simvastatin, having previously been mixed with 20 g of lactose monohydrate in a suitable high velocity mixer for about 1 minute. The wetting/drying operation was repeated until the powder mix was used up.

The capsules obtained were analysed using the method aforegiven in example 1: release of simvastatin after 30 minutes was found to be 96%, while the capsules containing the fatty acid mixture were found to be gastroresistant.

### Example 15

55 g of a 10% PVP solution in isopropyl alcohol were nebulized onto the remaining 1 Kg of gastroresistant capsules prepared as aforedescribed in example 13, followed by application of 17 g of simvastatin, having previously been mixed with 19 g of lactose monohydrate in a suitable high velocity mixer for about 1 minute. The wetting/drying operation was repeated until the powder mix was used up.

The capsules obtained were analysed using the method aforegiven in example 1: release of simvastatin after 30 minutes was found to be 96%, while the capsules containing the fatty acid mixture were found to be gastroresistant.

### Example 16

Under the same operative conditions as in example 1, 550 g of a 5% solution of HPMC P-50 in 70:30 acetone:ethanol were nebulized onto 1 Kg of soft gelatin capsules containing about 500 mg per capsule of the fatty acid mixture of example 1 with the purpose of rendering the capsules gastroresistant: A solution having the following composition:

| | |
|---|---|
| 10% PVP in isopropyl alcohol | 50.0 g |
| Butylhydroxyanisole | 0.04 g |
| 10% Eudragit E in isopropyl alcohol | 100.0 g |
| Isopropyl alcohol | 50.0 g |

was then nebulized onto said capsules. The following powder mix was subsequently applied:

| | |
|---|---|
| Simvastatin | 17.0 g |
| Lactose monohydrate | 17.0 g |
| Ascorbic acid | 4.3 g |
| Citric acid | 2.1 g |

The wetting/drying operation was repeated until the powder mix was used up. The capsules obtained were additionally coated with 400 g of a membrane based on 5% HPMC in ethanol with the aim of conferring further protection to the active principle, rendering it more stable.

The capsules obtained were analysed using the method aforegiven in example 1: is release of simvastatin after 30 minutes was found to be 96%, while the capsules containing the fatty acid mixture were found to be gastroresistant.

### Example 17

Under the same operative conditions as in example 1, a solution with the following composition:

| | |
|---|---|
| 10% PVP in isopropyl alcohol | 50.0 g |
| Butylhydroxyanisole | 0.04 g |
| 10% Eudragit E in isopropyl alcohol | 100.0 g |
| Isopropyl alcohol | 50.0 g |

was nebulized onto 1 Kg of soft gelatin capsules containing about 500 mg per capsule of the fatty acid mixture of example 1.

The following powder mix was subsequently applied:

| | |
|---|---|
| Simvastatin | 17.0 g |
| Lactose monohydrate | 17.0 g |
| Ascorbic acid | 4.3 g |
| Citric acid | 2.1 g |

The wetting/drying operation was repeated until the powder mix was used up. The capsules obtained were additionally coated with 400 g of a membrane based on 5% HPMC in ethanol with the aim of conferring further protection to the active principle, rendering it more stable.

The capsules obtained were analysed using the method aforegiven in example 1: the quantity of degradation products was found to be less than 1% and the simvastatin content was 10 mg per capsule.

### Example 18

Under the same operative conditions as in example 1, 550 g of a 5% solution of HPMC P-50 in 70:30 acetone:ethanol were nebulized onto 1 Kg of soft gelatin capsules containing about 500 mg per capsule of the fatty acid mixture of example 1 with the purpose of rendering the capsules gastroresistant. A solution having the following composition:

| | |
|---|---|
| 10% PVP in isopropyl alcohol | 50.0 g |
| Butylhydroxyanisole | 0.04 g |
| 10% Eudragit E in isopropyl alcohol | 100.0 g |
| Isopropyl alcohol | 50.0 g |

was then nebulized onto said capsules. The following powder mix was subsequently applied:

| | |
|---|---|
| 50% microencapsulated simvastatin | 34.0 g |
| Lactose monohydrate | 17.0 g |
| Ascorbic acid | 4.3 g |
| Citric acid | 2.1 g |

The wetting/drying operation was repeated until the powder mix was used up. The capsules obtained were additionally coated with 400 g of a membrane based on 5% HPMC in ethanol with the aim of conferring further protection to the active principle, rendering it more stable.

The capsules obtained were analysed using the method aforegiven in example 1: release of simvastatin after 30 minutes was found to be 96%, while the capsules containing the fatty acid mixture were found to be gastroresistant.

### Example 19

Under the same operative conditions as example 1, a suspension with the following composition:

| | | |
|---|---|---|
| Simvastatin | 17.0 | g |
| Lactose monohydrate | 15.0 | g |
| Ascorbic acid | 8.0 | g |
| Vitamin E | 15.0 | g |
| 20% PVP in isopropyl alcohol | 50.0 | g |
| Isopropyl alcohol | 200.0 | g |

was nebulized onto 1 Kg of soft gelatin capsules containing about 500 mg per capsule of the fatty acid mixture of example 1. The capsules thus obtained were then coated with a film membrane having the following composition:

| | |
|---|---|
| 20% PEG 4000 in isopropyl alcohol | 100.0 g |
| Isopropyl alcohol | 150.0 g |

The capsules obtained were analysed using the method aforegiven in example 1: release of simvastatin after 30 minutes was found to be 96%,

### Example 20

Under the same operative conditions as example 1, a solution having the following composition:

| | |
|---|---|
| Vitamin E | 15.0 g |
| 20% PVP isopropyl alcohol | 90.0 g |
| 10% Eudragit E in isopropyl alcohol | 180.0 g |
| Isopropyl alcohol | 90.0 g |

was nebulized onto 1 Kg of soft gelatin capsules containing about 500 mg per capsule of the fatty acid mixture of example 1. The following blend of powders was subsequently applied:

| | |
|---|---|
| Simvastatin | 17.0 g |
| Lactose monohydrate | 15.0 g |

The wetting/drying operation was repeated until the powder mix was used up. The capsules thus obtained were further coated with a membrane having the following composition:

| | |
|---|---|
| 20% PEG 4000 in isopropyl alcohol | 25.0 g |
| Isopropyl alcohol | 25.0 g |
| 10% Eudragit E in isopropyl alcohol | 50.0 g |

The capsules obtained were analysed using the method aforegiven in example 1: release of simvastatin after 30 minutes was found to be 95%.

1. Pharmaceutical composition for the oral administration of omega polyenoic fatty acids and one or more active principles incompatible therewith, comprising a capsule containing one or more blended omega polyenoic fatty acids, and a film coating comprising one or more of said active principles incompatible therewith and one or more suitable filming agents, possibly mixed with at least one inert substance.

2. Pharmaceutical composition as claimed in claim 1, wherein said capsule is a soft or hard gelatin capsule.

3. Pharmaceutical composition as claimed in claim 1, wherein said omega polyenoic fatty acids are chosen from the group consisting of omega-3 polyenoic fatty acids, omega-6 polyenoic fatty acids and blends thereof.

4. Pharmaceutical composition as claimed in claim 1, wherein said omega polyenoic fatty acids are omega-3 polyenoic fatty acid blends.

5. Pharmaceutical composition as claimed in claim 4, wherein said omega-3 polyenoic fatty acid blends contain a total quantity of EPA and DHA of between 20 and 98% by weight on the total weight of the blend.

6. Pharmaceutical composition as claimed in claim 5, wherein in said blends the total quantity of EPA and DHA is at least 60% by weight on the total weight of the blend.

7. Pharmaceutical composition as claimed in claim 5, wherein in said blends the weight ratio of EPA to DHA is between 0.05 and 2.5.

8. Pharmaceutical composition as claimed in claim 7, wherein in said blends the weight ratio of EPA to DHA is between 0.9 and 1.5.

9. Pharmaceutical composition as claimed in claim 1, wherein said active principles incompatible with omega polyenoic fatty acids are chosen from statins and platelet anti-aggregants.

10. Pharmaceutical composition as claimed in claim 9, wherein said statin is simvastatin and said anti-aggregant is acetylsalicylic acid.

11. Pharmaceutical composition as claimed in claim 1, wherein said active principles incompatible with omega polyenoic fatty acids consist of simvastatin, either alone or mixed with an additional active principle incompatible with said fatty acids.

12. Pharmaceutical composition as claimed in claim 10, wherein said simvastatin is in the form of a pure raw material or in the form of a microencapsulate comprising from 10 to 90% of simvastatin.

13. Pharmaceutical composition as claimed in claim 11, wherein said additional active principle is chosen from the group consisting of butyl hydroxyanisole, citric acid, vitamin E, ascorbic acid and mixtures thereof.

14. Pharmaceutical composition as claimed in claim 1, wherein said capsule contains from 100 to 1,500 mg of omega polyenoic fatty acids and said film coating comprises acetylsalicylic acid in a quantity between 10 and 300 mg.

15. Pharmaceutical composition as claimed in claim 1, wherein said capsule contains from 100 to 1,500 mg of omega polyenoic fatty acids and said film coating comprises simvastatin in a quantity from 10 to 160 mg, either alone or mixed with an additional active principle incompatible with said fatty acids.

16. Pharmaceutical composition as claimed in claim 1, also comprising excipients and/or pharmaceutically acceptable diluents.

17. Pharmaceutical composition as claimed in claim 1, also comprising at least one additional film coating free of said active principles and comprising one or more suitable filming-agents, possibly mixed with at lease one inert substance.

18. Pharmaceutical composition as claimed in claim 17, wherein said filming-agents are chosen from hydroxypropyl methyl cellulose, polyvinylpyrrolidone, polyvinyl alcohol-polyethylene glycol copolymers, basic polymethacrylate and mixtures thereof.

19. Pharmaceutical composition as claimed in claim 17, wherein said inert substance is chosen from talc and lactose monohydrate.

20. Pharmaceutical composition as claimed in claim 1, also comprising at least one gastroresistant coating on said capsule and/or over said film coating, comprising one or more suitable agents sensitive to pH variations, preferably chosen from methacrylic acid derivatives, hydroxypropyl methyl cellulose succinate, hydroxymethyl cellulose phthalate, cellulose acetate phthalate and mixtures thereof.

21. Pharmaceutical composition as claimed in claim 1, also comprising, over said film coating, at least one coating suitable for modified release of the active principles and comprising one or more suitable agents not sensitive to pH variations preferably chosen from ethyl cellulose, cellulose acetate butyrate, methacrylic acid derivatives, Shellac and mixtures thereof.

22. Process for preparing a pharmaceutical composition as defined in claim 1, comprising the following steps:
i) preparing the capsule containing said polyenoic fatty acids;
ii) preparing a solution or suspension comprising at least one film-forming agent and a suitable solvent, said active principle or a mixture of active principles incompatible with said fatty acids, and possibly one or more inert substances;
iii) applying the film coating onto the capsule derived from step i), by nebulizing the solution or suspension prepared in step ii).

23. Process for preparing a pharmaceutical composition as defined in claim 1, comprising the following steps:
i′) preparing the capsule containing said omega polyenoic fatty acids;
ii′) preparing a solution or suspension comprising at least one film-forming agent and a suitable solvent;
iii′) possibly mixing said active principle or mixture of active principles in powder form incompatible with said fatty acids, with said inert substance possibly present;
iv′) applying said film coating onto the capsule derived from step i′) by several alternate phases of nebulizing the solution or suspension prepared in step ii′) and distributing said active principle or mixture of active principles in powder form, incompatible with said fatty acids, possibly mixed with said inert substance as prepared in step iii′).

24. Process as claimed in claim 22, wherein said suitable solvents are chosen from acetone, isopropyl alcohol and mixtures thereof, possibly mixed with a buffer at pH 4-8 if said second active principle or active principle mixture is simvastatin, whereas they are chosen from water, ethanol and mixtures thereof, possibly mixed with a buffer at pH 4-8, if said second active principle or active principle mixture is acetylsalicylic acid.

25. Process as claimed in claim 23, wherein said suitable solvents are chosen from acetone, isopropyl alcohol and mixtures thereof, possibly mixed with a buffer at pH 4-8 if said second active principle or active principle mixture is simvastatin, whereas they are chosen from water, ethanol and mixtures thereof, possibly mixed with a buffer at pH 4-8, if said second active principle or active principle mixture is acetylsalicylic acid.

26. Process as claimed in claim 24, wherein said buffer at pH 4-8 is chosen from acetate and phosphate buffers.

27. Process as claimed in claim 22, wherein at steps ii) or ii′) said filming-agent is firstly dissolved in the pre-selected solvent, then mixed with the other components in solution or suspension.

28. Process as claimed in claim 23, wherein at steps ii) or ii′) said filming-agent is firstly dissolved in the pre-selected solvent, then mixed with the other components in solution or suspension.

29. Process as claimed in claim 22, wherein said filming-agent is used in a quantity between 1 and 20% by weight on the total weight of the solution or suspension, while said inert substance, if present, is added in a quantity between 2 and 30% by weight on the total weight of the solution or suspension.

30. Process as claimed in claim 23, wherein said filming-agent is used in a quantity between 1 and 20% by weight on the total weight of the solution or suspension, while said inert substance, if present, is added in a quantity between 2 and 30% by weight on the total weight of the solution or suspension.

31. Process as claimed in claim 29, wherein said filming-agent is used in a quantity of 5% by weight and said inert substance in a quantity of 2% by weight.
